(19) United States
(12) Patent Application Publication
(10) Pub. No.: US 2008/0255365 A1
(43) Pub. Date: Oct. 16, 2008

(54) Microparticulate form of a Tetrahydropyridine derivative

(75) Inventors: Antoine CARON, Montbazin (FR); Jean-Pierre CHAMBON, Prades Le Lez (FR); Olivier MONNIER, Villeveyrac (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/138,582

(22) Filed: Jun. 13, 2008

Related U.S. Application Data
(63) Continuation of application No. 10/177,384, filed on Jun. 21, 2002, now abandoned.
Application No. 10/177,384 is a continuation of application No. 09/331,514, filed on Jun. 22, 1999, now abandoned.

(30) Foreign Application Priority Data
Dec. 23, 1996 (FR) 9615905

(51) Int. Cl. C07D 213/04

(52) U.S. Cl. 546/346

(57) ABSTRACT

The invention relates to a microparticulate form of 1-[2-(2-naphthyl)-ethyl]-4-(3-trifluoromethylphenyl)-1,2,3,6-tetrahydropyridine hydrochloride consisting of particles for which at least 55% of the population have a diameter below 50 micrometers, and to pharmaceutical compositions in which it is present.

This application is a continuation of U.S. application Ser. No. 10/177,384, filed Jun. 21, 2002, now pending, which is a continuation of U.S. application Ser. No. 09/331,514, filed Jun. 22, 1999, now abandoned, which was the National Stage of International application No. PCT/FR97/02,394, filed Dec. 23, 1997, all of which are incorporated herein by reference in their entirety; which claims the benefit of priority of French Patent Application No. 96/15,905, filed Dec. 23, 1996.

The present invention relates to a microparticulate form of 1-[2-(2-naphthyl)ethyl]-4-(3-trifluoromethylphenyl)-1,2,3,6-tetrahydropyridine hydrochloride.

1-[2-(2-Naphthyl)ethyl]-4-(3-trifluoromethylphenyl)-1,2,3,6-tetrahydropyridine, hereafter designated by its code number SR 57746, and its pharmaceutically acceptable salts were first described in EP 0 101 381 as anorexigenic agents and subsequently as antianxiodepressants (U.S. Pat. No. 5,026,716), anticonstipation agents (U.S. Pat. No. 5,109,005), neurotrophic agents (U.S. Pat. No. 5,270,320), free radical scavengers (U.S. Pat. No. 5,292,745) and cardioprotective agents (U.S. Pat. No. 5,378,709).

EP 0 101 381 describes SR 57746 in the form of the hydrochloride, hereafter called SR 57746 A, and this salt was used in preclinical and clinical trials on healthy volunteers (Phase I). According to said document, SR 57746 is isolated by crystallization from ethanol, especially absolute ethanol.

In the preclinical trials, especially in the animal pharmacology and toxicology tests, SR 57746 showed a constant activity and behavior. Likewise, the pharmacokinetic studies on animals gave constant and reproducible results.

By contrast, in the clinical trials carried out on healthy volunteers, SR 57746 A, administered orally, was found to show a high variability in the plasma concentrations and the pharmacodynamic effects of the active principle.

In the first clinical trials on patients suffering from very serious diseases, especially amyotrophic lateral sclerosis, the dose of SR 57746 A was kept very low, namely 2 mg/day, at which dose the product proved promising (W. G. Bradley, paper entitled “New drugs for amyotrophic lateral sclerosis”, American Academy of Neurology meeting, Mar. 23-30, 1996; pages 240-23/240-28).

It has furthermore been found that the preparation of larger amounts of SR 57746 A by the method of isolation described in EP 0 101 381 does not successfully yield a product with constant characteristics which makes it possible to overcome the disadvantages noted in the Phase I clinical trials.

It was found more particularly that, by the method of isolation described in EP 0 101 381, the SR 57746 A obtained consists of crystals whose size is not constant and specifically is greater than 150 micrometers; more particularly, it is 150-600 micrometers for at least about 75% of the crystals.

It has now been found that when SR 57746 A is isolated by recrystallization from absolute ethanol, with stirring, the SR 57746 A obtained is formed of crystals for which at least 55% of the population have a size below 50 micrometers, and that the resulting product possesses a higher activity when administered orally in human clinical trials.

It has also been found that by atomizing a solution of SR 57746 A in ethanol optionally containing water, the active principle is obtained in an essentially amorphous form which has a constant absorption level in man and a very high activity, enabling the active principle to be administered in very low dosages.

It has also been found that said atomization gives small spherical particles with a diameter below 15 micrometers in a constant and reproducible manner, making it possible to overcome the disadvantages due to the variability of the characteristics of the SR 57746 A isolated as described in EP 0 101 381.

Finally, it has been found that an identical result is obtained by micronization of the SR 57746 A obtained by crystallization from absolute ethanol, as described in EP 0 101 381, giving crystals with a size below 50 micrometers.

Thus, according to one of its aspects, the present invention relates to a microparticulate form of 1-[2-(2-naphthyl)ethyl]-4-(3-trifluoromethylphenyl)-1,2,3,6-tetrahydropyridine hydrochloride consisting of microparticles for which at least 55% of the population have a diameter below 50 micrometers.

The microparticles according to the present invention can be microspheres obtainable by atomization or microcrystals obtained by screening or micronization.

The expression “diameter below 50 micrometers” refers to both the microspheres and the microcrystals, the latter being comparable to microspheres.

The size of the microparticles according to the present invention advantageously corresponds to a diameter below 25 micrometers, preferably below 15 micrometers. Microparticles of which the majority (80-85%) have a diameter below 10 micrometers are particularly preferred.

An SR 57746 A of fine particle size, namely a product formed of a population of crystals for which at least 55% have a size below 50 micrometers, can be prepared by recrystallization of the product obtained according to EP 0 101 381, wherein said product is heated in absolute ethanol, with stirring, heating is stopped when dissolution is complete and stirring is stopped when the temperature reaches about 40° C., the mixture is left to stand for 16 to 60 hours at room temperature and then stirred vigorously at 10-18° C. and the product is filtered off and dried.

Alternatively, an SR 57746 A of the same fine particle size can be obtained by following the procedure described in EP 0 101 381, by reacting 4-(3-trifluoromethylphenyl)-1,2,3,6-tetrahydropyridine with 2-(2-chloroethyl)naphthalene in the presence of triethylamine or by reducing 1-(2-naphthylacetyl)-4-(3-trifluoromethylphenyl)-1,2,3,6-tetrahydropyridine with lithium aluminum hydride, but then taking up the residue, consisting of 1-[2-(2-naphthyl)ethyl]-4-(3-trifluoromethylphenyl)-1,2,3,6-tetrahydropyridine base, directly with hydrochloric acid in absolute ethanol under reflux and then following the procedure illustrated above.

The microparticles according to the present invention can also be prepared by atomizing solutions of SR 57746 A, advantageously in (C₁-C₃)alkanols, (C₃-C₆)alkanones or ethyl acetate, optionally in the presence of water, and preferably by atomizing a solution of SR 57746 A in ethanol containing from 0 to 40% of water, in a conventional atomizer, for example a Büchi mini spray dryer, the pump capacity, suction, heating and flow rate being adjusted so as to establish an inlet temperature of between 150 and 190° C., an outlet temperature of between 50 and 120° C. and a partial vacuum of 30 to 70 mbar.

Atomization of these solutions gives small spherical particles with a size below 50 micrometers, 80-85% of which, in particular, can have a diameter below 10 micrometers, and which, in differential scanning calorimetry (DSC) carried out using a Perkin Elmer DSC7 apparatus calibrated relative to indium and cyclohexane, show a single broad peak from 130 to 160° C. with a maximum at 146±3° C.

The microparticles according to the present invention are advantageously prepared by micronization of the SR 57746 A obtained as described in EP 0 101 381. This micronization can be carried out in a conventional apparatus for obtaining microcrystals with a size below 50 micrometers, for example in an ALPINE 200 AS micronizer, the SR 57746 A being introduced into the micronization chamber (diameter of 200 mm) at a rate of 15 to 50 kg/hour and a working pressure of 1 to 6.5 bar, and the product being recovered in a filter bag.

Particularly advantageously, the operating conditions are such that the microcrystals obtained have a population of particles with a mean size below 25 micrometers or, preferably, below 15 micrometers. Preferably, the operating conditions are such that 80-85% of the population of microcrystals obtained have a size below 10 micrometers.

If the microcrystals obtained by this procedure tend to aggregate, the aggregates can be screened prior to preparation of the pharmaceutical compositions. However, any aggregation of the microcrystals does not change the absorption of the active principle, as demonstrated in the CACO-2 cell test illustrated below.

To avoid such aggregation, the SR 57746 A can optionally be micronized in the presence of mannitol, for example, and preferably D-mannitol.

As indicated above, the microparticles according to the present invention possess properties which make them particularly advantageous for the preparation of the pharmaceutical compositions in which they are present.

More particularly, it has been demonstrated that the microcrystalline form not only makes it possible to reduce the dosage amount present in the pharmaceutical compositions, but also, in particular, makes it possible to render the oral absorption uniform and thus to have a constant therapeutic response in every patient. Moreover, said absorption is independent of food conditions.

A study concerning the determination of the in vitro absorption of the microparticles according to the present invention was carried out using the CACO-2 monolayer model. This test, which is widely used as a predictive intestinal epithelial model for drug absorption (P. Artusson, Crit. Rev. Ther. Drug, 1991, 8: 305-330), made it possible to show significant differences in terms of dissolution and permeability between micronized SR 57746 A and non-micronized non-atomized SR 57746 A.

The results obtained show that, in the medium used (Hanks' solution supplemented with 10% of fetal calf serum and taurocholic acid), the rates of dissolution and permeability are significantly different for micronized or atomized SR 57746 A and for non-micronized non-atomized SR 57746 A. More particularly, it was demonstrated that the dissolution and permeability are normalized—i.e. rendered uniform—after micronization or atomization.

The results obtained in vitro were confirmed in vivo by comparing the observations made in two clinical trials on healthy volunteers, the first trial evaluating the effect of food on the oral absorption of the SR 57746 A obtained according to EP 0 101 381, and the second trial evaluating the effect of food on the oral absorption of the SR 57746 A of Example 5 below. In both the trials, the criterion for evaluation of the absorption was the area under the curve of the plasma SR 57746 concentrations as a function of time.

Analysis of the results showed that:

when the product is administered with a meal, the required dose of SR 57746 A prepared according to EP 0 101 381 is three to four times greater than that of the product of Example 5 below in order to obtain the same absorption;
when the product is administered on an empty stomach, the required dose of SR 57746 A prepared according to EP 0 101 381 is about nine times greater than that of the product of Example 5 below in order to obtain the same absorption.

It was found in these trials that, surprisingly, in the case of administering the SR 57746 A prepared according to EP 0 101 381, the absorption is two to three times greater when the product is taken with a meal, whereas in the case of administering the product of Example 5, the absorption is the same, whether the product be administered on an empty stomach or with food.

These results demonstrate the value of the present invention, which makes it possible to provide a product having a better absorption which is not influenced by the intake of food.

Thus, according to another of its aspects, the present invention relates to pharmaceutical compositions containing, as the active principle, a microparticulate form of 1-[2-(2-naphthyl)ethyl]-4-(3-trifluoromethylphenyl)-1,2,3,6-tetrahydropyridine hydrochloride consisting of microparticles for which at least 55% of the population have a size below 50 micrometers, advantageously below 25 micrometers and preferably, for 80-85% of the particles, below 10 micrometers.

The amount of active principle to be administered depends on the nature and severity of the diseases to be treated and on the weight of the patients. Nevertheless, the amount of active principle present in the dosage unit can be from 0.1 to 5 mg, advantageously from 0.5 to 3 mg and preferably 2 mg (calculated as the free base). The preferred unit doses will generally comprise 0.5, 1, 1.5, 2, 2.5 or 3 mg (calculated as the free base) of micronized product.

These unit doses will normally be administered one or more times a day, for example once or twice a day, the overall dose in man varying between 0.2 and 10 mg per day, advantageously between 1 and 6 mg per day (calculated as the free base).

In the pharmaceutical compositions of the present invention, the active principle can be administered to animals and humans in unit forms of administration, mixed with conventional pharmaceutical carriers, for the treatment of the diseases indicated in patents U.S. Pat. Nos. 5,026,716, 5,109,005, 5,270,320, 5,292,745 and 5,378,709, and especially for the treatment of neurodegeneration. The appropriate unit forms of administration include tablets, which may be divisible, gelatin capsules, powders and granules.

When preparing a solid composition in the form of tablets, the active principle is mixed with a pharmaceutical vehicle such as gelatin, starch, lactose, magnesium stearate, talcum, gum arabic or the like. The tablets can be coated with sucrose or other appropriate substances, or else they can be treated so as to have a prolonged or delayed activity and so as to release a predetermined amount of active principle continuously.

A preparation in the form of gelatin capsules is obtained by mixing the active ingredient with a diluent and pouring the resulting mixture into soft or hard gelatin capsules.

The active principle can also be formulated as microcapsules, optionally with one or more carriers or additives.

In the pharmaceutical compositions according to the present invention, the active principle can also be in the form of an inclusion complex in cyclodextrins, their ethers or their esters.

The compositions of the invention can also be prepared by an extrusion-spheroidization method, which makes it possible to obtain spheroids of the desired size. In this method, the microparticulate SR 57746 A, preferably atomized or micronized, is mixed with the excipients and demineralized water, the resulting mass is granulated and extruded to give an extrusion mass which flows freely through orifices of the desired diameter, the extrudate is spheroidized to give spheroids of the same diameter as the orifices, and the resulting spheroids are dried and, preferably, introduced into gelatin capsules. In this manner, the SR 57746 A and the excipients are mixed so as to give a ready-to-use pharmaceutical composition.

The Examples which follow illustrate the invention.

### EXAMPLE 1

Operating under the conditions described in Example 1 of EP 0 101 381, 4-(3-trifluoromethylphenyl)-1,2,3,6-tetrahydropyridine is reacted with 2-(2-chloroethyl)naphthalene in ethanol under reflux, in the presence of triethylamine, for 24 hours. The mixture is concentrated to dryness, the residue is taken up with ethyl ether and the ether solution which is filtered and washed with water, is dried and evaporated.

1-[2-(2-Naphthyl)ethyl]-4-(3-trifluoromethylphenyl)-1,2,3,6-tetrahydropyridine hydrochloride is subsequently isolated in the following manner: The residue is taken up with hydrochloric acid in 100 ethanol and the mixture is refluxed, with stirring. When dissolution is complete, the heating is stopped and the solution is allowed to cool, with stirring. After about ten minutes, the stirring is stopped and the mixture is left to stand at room temperature for 48 hours. The precipitate is filtered off and washed with absolute ethanol and the cake is made into a paste again in absolute ethanol, with pneumatic stirring, filtered off and dried at 40° C. under vacuum.

This gives, with an overall yield of 10%, an SR 57746 A whose particle size distribution is given in Table I.

TABLE I

| Size in micrometers | Percentage |
|---|---|
| 4.0-6.0 | 0.8 |
| 6.0-8.0 | 2.6 |
| 8.0-10.0 | 3.8 |
| 10.0-14.0 | 6.3 |
| 14.0-20.0 | 6.4 |
| 20.0-30.0 | 13.9 |
| 30.0-40.0 | 15.8 |
| 40.0-50.0 | 9.6 |
| 50.0-60.0 | 4.9 |
| 60.0-70.0 | 3.4 |
| 70.0-80.0 | 1.8 |
| 80.0-90.0 | 1.9 |
| 90.0-100.0 | 1.8 |
| 100.0-150.0 | 8.1 |
| 150.0-200.0 | 6.2 |
| 200.0-300.0 | 7.5 |
| 300.0-400.0 | 3.6 |
| 400.0-500.0 | 1.6 |
| 500.0-600.0 | 0.1 |

The resulting microparticulate form of SR 57746 A contains 59.2% of particles with a size below 50 micrometers.

### EXAMPLE 2

A mixture of 636 g of SR 57746 A, obtained as described in EP 0 101 381 and formed of crystals of which 77% have a size of between 150 and 600 micrometers, with 5 volumes of absolute ethanol is refluxed, with stirring, until the product has completely dissolved, the heating is then stopped and, when the temperature reaches 40° C., the stirring is stopped and the mixture is left to stand for 16 hours at room temperature. It is brought to 16° C., with vigorous stirring, and, after 10-20 minutes under these conditions, the product is filtered off and dried under vacuum at 40° C. for 24 hours. This gives 415 g of SR 57746 A consisting of a population of microparticles of which 60.3% have a size below 50 micrometers.

### EXAMPLE 3

A solution of 3 g of SR 57746 A in 300 ml of ethanol is atomized in a Büchi mini spray dryer apparatus according to the principle of parallel-flow nozzle atomization, the pump capacity, suction, heating and flow rate being adjusted so as to have an inlet temperature of 172° C., an outlet temperature of 107° C. and a partial vacuum of 40 mbar. Under these conditions, the product having a single broad peak in DSC with a maximum at 145° C. is obtained. The particles obtained are spherical and the mean size of the very homogeneous population does not exceed 5 micrometers.

### EXAMPLE 4

A solution of 3 g of SR 57746 A in 210 ml of ethanol and 90 ml of water is atomized in the apparatus described in Example 3 according to the principle of parallel-flow nozzle atomization, the pump capacity, suction, heating and flow rate being adjusted so as to have an inlet temperature of 172° C., an outlet temperature of 63° C. and a partial vacuum of 60 mbar. Under these conditions, an essentially amorphous, atomized SR 57746 A is obtained which, in the DSC thermogram, showed a single broad peak with a maximum at 147.6° C. The particles obtained are spherical and the mean size of the very homogeneous population does not exceed 5 micrometers.

### EXAMPLE 5

24 kg of SR 57746 A are introduced into the micronization chamber (diameter 200 mm) of an ALPINE 200 AS micronizer at a rate of 25 kg/hour and at a working pressure of 6.5 bar and the thereby micronized product is recovered in a filter bag. This gives a micronized SR 57746 A with a particle size distribution such that all the particles have a size below 20 micrometers and 85% of the particles have a size below 10 micrometers.

### EXAMPLE 6

Pharmaceutical composition containing, as the active principle, the micronized SR 57746 A according to Example 5 above:

| | |
|---|---|
| Active principle | 2.192 mg |
| Corn starch | 141.218 mg |
| Anhydrous colloidal silica | 0.200 mg |
| Magnesium stearate | 0.400 mg |

The active principle is screened at 0.2 mm and then premixed with the excipients. This mixture is screened at 0.315 mm, remixed and then screened again at 0.315 mm. After a final mixing, the composition is introduced into no. 3 gelatin capsules at a rate of 170 mg of composition containing an amount of micronized SR 57746 A which corresponds to 2 mg of 1-[2-(2-naphthyl)ethyl]-4-(3-trifluoromethylphenyl)-1,2,3,6-tetrahydropyridine base.

What is claimed is:

1. An oral dosage unit comprising a microparticulate form of 1-[2-(2-naphthyl)ethyl]-4-(3-trifluoromethylphenyl)-1,2,3,6-tetrahydropyridine hydrochloride consisting of particles wherein at least 55% of said particles have a diameter below 50 micrometers, wherein said microparticles exhibit a better absorption which is not influenced by the intake of food and wherein absorption of said microparticles is at least two times more than the non-micronized form of 1-[2-(2-naphthyl)ethyl]-4-(3-trifluoromethylphenyl)-1,2,3,6-tetrahydropyridine hydrochloride.

2. The dosage unit according to claim 1 wherein the particle diameter is below 25 micrometers.

3. The dosage unit according to claim 2 wherein the particle diameter is below 15 micrometers.

4. The dosage unit according to claim 3 wherein the diameter of 80-85% of the population of particles is below 10 micrometers.

5. The dosage unit according to claim 1 wherein the particles are microspheres.

6. The dosage unit according to claim 5 wherein the microparticles consist of 1-[2-(2-naphthyl)ethyl]-4-(3-trifluoromethylphenyl)-1,2,3,6-tetrahydropyridine hydrochloride in an essentially amorphous form.

7. The dosage unit according to claim 1 wherein the particles are micronized crystals.

8. A pharmaceutical composition containing a microparticulate form according to claim 1 as the active principle.

9. A composition according to claim 8 wherein it is in the form of a tablet.

10. A composition according to claim 9 wherein each tablet contains from 0.1 to 5 mg of active principle (calculated as the free base).

11. A composition according to claim 10 wherein each tablet contains from 0.5 to 3 mg of active principle (calculated as the free base).

12. A composition according to claim 11 wherein each tablet contains 2 mg of active principle (calculated as the free base).

13. A pharmaceutical composition containing a microparticulate form according to claim 2 as the active principle.

14. A pharmaceutical composition containing a microparticulate form according to claim 3 as the active principle.

15. A pharmaceutical composition containing a microparticulate form according to claim 4 as the active principle.

16. A pharmaceutical composition containing a microparticulate form according to claim 5 as the active principle.

17. A pharmaceutical composition containing a microparticulate form according to claim 6 as the active principle.

18. A pharmaceutical composition containing a microparticulate form according to claim 7 as the active principle.
